(19) United States
(12) Patent Application Publication
(10) Pub. No.: US 2021/0128897 A1
(43) Pub. Date: May 6, 2021

(54) BONE GROWTH STIMULATOR AND METHODS OF USE

(71) Applicant: NEW YORK UNIVERSITY, New York, NY (US)

(72) Inventors: Mani ALIKHANI, Hoboken, NJ (US); Cristina TEIXEIRA, New York, NY (US); Chinapa SANGSUWON, New York, NY (US); Sarah ALANSARI, New York, NY (US); Serafim OLIVEIRA, Viseu (PT); Candido ROQUE, Tondela (PT)

(21) Appl. No.: 16/639,404

(22) PCT Filed: Aug. 17, 2018
(86) PCT No.: PCT/US2018/046900

§ 371 Date: Feb. 14, 2020

Related U.S. Application Data
(60) Provisional application No. 62/546,799, filed on Aug. 17, 2017.

(51) Int. Cl. A61M 37/00; A61C 19/06

(52) U.S. Cl. CPC A61M 37/0015; A61C 19/063; A61M 2202/07; A61M 2037/0023; A61M 2037/003

(57) ABSTRACT

The present invention relates to a device. The device includes an elongate handle having a proximal end and a distal end; a head portion extending from the proximal end of the elongate handle, the head portion having a first interior-facing surface and an opposing second exterior-facing surface; and a plurality of microneedles extending from, and perpendicular to, the first interior-facing surface of the head portion. Each of the plurality of microneedles has a base connected to the first interior-facing surface of the head portion and a free tip end. The present invention also relates to a method for stimulation of cortical bone formation using the device of the present invention.

This application claims priority benefit of U.S. Provisional Patent Application No. 62/546,799, filed Aug. 17, 2017, which is hereby incorporated by reference in its entirety.

### FIELD OF THE INVENTION

The present invention relates to devices and methods of their use in stimulating bone formation.

### BACKGROUND OF THE INVENTION

One of the main challenges in orthodontics relates to the cortical bone that surrounds the alveolar bone. This bone acts as an envelope that determines the limit to which a tooth can be moved safely. Since cortical bone does not follow the moving tooth, any movement towards that cortical plate may push the tooth out of the bone envelope. This may cause bone loss around the tooth, which can jeopardize the long term prognosis of the tooth. To compensate for this limitation, many extensive and expensive surgical approaches have been proposed to cut the bone and tooth together as a segment and move the tooth and the surrounding cortical bone surgically. Due to the complexity of these procedures, many adults prefer to not go through surgery and therefore never accomplish proper orthodontic correction.

In addition, the major limitation of orthodontic corrections in patients with mild to moderate skeletal deformities is the inability to move teeth beyond the confines of the cortical bone to compensate for the jaws discrepancy and provide a functional occlusion. Therefore, there is no alternative treatment modality to surgical correction of skeletal problems, leaving patients no option but to adopt less functional bites, sacrifice teeth for an incomplete dental compensation of jaws discrepancies, and settle for less than optimum treatment outcomes.

Skeletal problems can occur in three planes of space (vertical, sagittal, and transverse) and the prevalence can vary with ethnicity, being as high as 22.5% for class II and 12.6% for class III (see Silva et al. “Prevalence of Malocclusion Among Latino Adolescents,” Am. J. Orthod. Dentofacial Orthop. 119(3):313-315 (2001)), 3% for open bites, and even a larger percentage of the population (i.e., 9.1%) present narrow deficient jaws (see Brunelle et al., “Prevalence and Distribution of Selected Occlusal Characteristics in the US Population, 1988-1991,”J. Dent. Res. 75(2 Suppl.): 706-713 (1996)). To help the growing population of adults seeking orthodontic treatment, there is an enormous need for a technique that can stimulate bone formation at the surface of cortical bone non-invasively. Such a technique would dramatically expand the limit of orthodontic dental corrections.

The present invention is directed to overcoming these and other deficiencies in the art.

### SUMMARY OF THE INVENTION

One aspect of the present invention relates to a device. The device includes an elongate handle having a proximal end and a distal end; a head portion extending from the proximal end of the elongate handle, the head portion having a first interior-facing surface and an opposing second exterior-facing surface; and a plurality of microneedles extending from, and perpendicular to, the first interior-facing surface of the head portion. Each of the plurality of microneedles has a base connected to the first interior-facing surface of the head portion and a free tip end.

Another aspect of the present invention relates to a method for stimulation of cortical bone formation. The method involves providing a device. The device includes an elongate handle having a proximal end and a distal end; a head portion extending from the proximal end of the elongate handle, the head portion having a first interior-facing surface and an opposing second exterior-facing surface; and a plurality of microneedles extending from, and perpendicular to, the first interior-facing surface of the head portion. Each of the plurality of microneedles has a base connected to the first interior-facing surface of the head portion and a free tip end. The method also involves positioning the free tip end of said plurality of microneedles in contact with tissue of a subject, where cortical bone underlies the subject's tissue. The method further involves applying pressure to the device to cause the microneedles to penetrate the subject's tissue without penetrating the cortical bone underlying the subject's tissue, thereby stimulating formation of cortical bone underlying the subject's tissue.

The form of the skeleton is dictated by cortical bone, and the density of bone is dictated by both trabecular and cortical bone. While there are non-invasive methods to increase trabecular bone density, there are none that can change the shape or form of cortical bone without aggressive surgical intervention. As described infra, the present invention represents a new treatment methodology for treating skeletal defects using a minimally invasive method by stimulating craniofacial cortical bone formation in the desired direction. The ability to drift the cortical bone in a desired direction will expand the biological envelope, range, and distance to which teeth can be moved orthodontically allowing non-surgical correction of severe malocclusions resulting from severe skeletal deformities.

The device and method of the present invention may be used to stimulate cortical bone formation at the surface of cortical bone (e.g., craniofacial bone), restore bone shape in bone deformity and deficient areas, stimulate new bone formation and cortical drift in the direction of force on bone (e.g., in the direction of tooth movement or jaw expansion), expand the range of tooth movement and orthopedic corrections in growing and non-growing subjects, and change the shape of the craniofacial bones to improve facial aesthetics. Corrections made possible with the device and method of the present invention include expansion of jaws, vertical jaw development, sagittal correction of dentoalveolar discrepancies, modelling of facial bones, and correction of bone defects due to syndromes (cleft palate) or pathology (periodontal disease, extractions). Currently, orthopedic corrections are an accepted treatment modality only for growing patients. The present invention establishes a new field of adult dentofacial orthopedic treatment in non-growing patients.

### BRIEF DESCRIPTION OF THE DRAWINGS

FIGS. 1A-1F are schematic illustrations of embodiments of a device according to the present invention. FIG. 1A is a side perspective view of a first embodiment of a device according to the present invention. FIGS. 1B and 1C are bottom and side views, respectively, of a second embodiment of a device according to the present invention. FIGS. 1D and 1E are cross-sectional, side views of two additional embodiments of the present invention including a chamber (fluid reservoir or compartment) within the device. FIG. 1F is a cross-sectional, side view of a further embodiment of a device according to the present invention including a chamber (fluid reservoir or compartment) within the device and a loading channel (exterior port) that permits filling of the compartment with a composition including, e.g., exogenous growth factors.

FIGS. 2A-2C show an embodiment of manually loading a composition including, e.g., exogenous growth factors into a compartment within a device of the present invention (FIGS. 2A-2B) via a port (or channel) suitable to receive a pipette tip (shown), needle tip, or the like. This embodiment facilitates injection of the composition (including, e.g., exogenous growth factors) to the area of interest by, e.g., applying pressure to the device to thereby expel the composition through microneedles of the device (FIG. 2C, arrow representing pressure).

FIGS. 3A and 3B are schematic illustrations of a further embodiment of a device according to the present invention. This embodiment is similar to the embodiment shown in FIGS. 2A-2C, but does not include a port for manual loading by, e.g., pipette or needle. This device may be pre-loaded (e.g., in manufacturing the device) with the composition of interest to facilitate the procedure for clinician. Like the embodiment shown in FIGS. 2A-2C, this embodiment allows delivery of the composition (including, e.g., exogenous growth factors) to the area of interest by, e.g., applying pressure to the device (shown with an arrow in FIGS. 3A and 3B) to expel the composition through microneedles of the device (shown in FIG. 3B).

FIGS. 4A-4D are schematic illustrations showing a method of using a device according to the present invention relative to the soft tissue and bone of an area of treatment of a subject. The device is positioned relative to the area of interest having soft tissue (e.g., gingiva and periosteum) with underlying cortical bone (FIG. 4A). Pressure is applied to the device suitable to cause microneedles to penetrate the subject's soft tissue without penetrating underlying cortical bone (FIGS. 4B and 4C) and the device is then withdrawn (FIG. 4D). This penetration of the soft tissue without penetration of the underlying cortical bone stimulates local release of endogenous growth factors under the periosteum (FIGS. 4C and 4D), which in turn stimulates cortical bone growth.

FIG. 5 is a schematic illustration showing the result of use of a device like that shown in FIGS. 3A and 3B, relative to the soft tissue and bone. In this embodiment, a method like that shown in FIGS. 4A-4D is employed with the additional step of (after penetration of the soft tissue and prior to removing the device) actuating a release mechanism (e.g., via pressure applied to the device, as shown in FIGS. 3A and 3B) to release a composition including, e.g., exogenous growth factors from a compartment in the head of the device through the microneedles and into the subject's tissue. As shown, this method therefore involves local injection of exogenous growth factors as well as stimulation of local production of endogenous growth factors.

FIG. 6 is a schematic representation of the effect of periosteal stimulation using a device according to the present invention and resultant stimulation of new cortical bone formation on the surface of the existing cortical bone.

FIGS. 7A and 7B are schematic illustrations showing changes in alveolar bone before expansion (FIG. 7A) and after expansion (FIG. 7B), illustrating of the effect of transverse orthodontic forces (F) on cortical bone in the absence of periosteal stimulation.

FIGS. 8A and 8B are schematic illustrations of the effect of orthodontic force (F) and periosteal stimulation (left panels of FIGS. 8A and 8B, small rectangles) in presence (FIG. 8B) and absence (FIG. 8A) of exogenous growth factor (left panel of FIG. 8B, teardrop shapes). FIG. 8A illustrates the effect of periosteal stimulation according to the present invention that stimulates endogenous release of growth factors on the surface of the cortical bone as the tooth moves in response to orthodontic forces. FIG. 8B illustrates the synergistic effect of periosteal stimulation according to the present invention that induces release of endogenous growth factors coupled with release of exogenous growth factors during application of orthodontic forces.

FIGS. 9A and 9B are a photograph (FIG. 9A) and micro-CT image (FIG. 9B) from an experimental subject illustrating the orthodontic transverse forces applied to the upper molars of the subject, pushing the molars towards the cortical bone.

FIGS. 10A and 10B are micro-CT images of control (no expansion) (FIG. 10A) and expansion (FIG. 10B) subjects collected after 56 days. In response to orthodontic forces, teeth were moved towards the cortical bone (FIG. 10B). As shown, the bone did not follow the tooth and, therefore, bone loss around the roots of the tooth was apparent (FIG. 10B, top panel) and the cortical plate became thinner (FIG. 10B, bottom panel).

FIGS. 11A and 11B are images illustrating the location of periosteal stimulation according to the method of the present invention in an experimental group. In addition to transverse orthodontic forces, animals were exposed to periosteal stimulation around the area of first and second molar (arrows in FIG. 11A and dots within the boxed area of FIG. 11B) once per week.

FIGS. 12A-12C are photographs of embodiments of devices according to the present invention similar to that used to apply periosteal stimulation, as described in the Examples herein.

FIGS. 13A and 13B are micro-CT images showing that, after 56 days of expansion treatment with periosteal stimulation according to methods of the present invention, the animals did not show any bone loss (FIG. 13A) and their cortical bone did not get thinner (FIG. 13B).

FIGS. 14A and 14B are fluorescent microscopy images showing significant increase in rate of bone formation in the area of cortical bone for the experimental group receiving expansion and periosteal stimulation (FIG. 14B) according to methods of the present invention, as compared to expansion alone (FIG. 14A).

FIGS. 15A-15C are fluorescent microscopy images showing results of a series of experiments in which animals were divided into 3 groups: 1) control that did not receive any treatment; 2) animals that received injection of PDGF once per week (around first molar area); and 3) animals that received periosteal stimulation according to methods of the present invention once per week in a similar area. No orthodontic force was applied to these animals/teeth. Compared to control (FIG. 15A), the group that received injection of PDGF did not show significant bone formation (FIG. 15B) while the group that received periosteal stimulation according to methods of the present invention demonstrated significant bone formation (FIG. 15C). These results indicate that the endogenous release of growth factors in response to periosteal stimulation according to methods of the present invention is more effective to stimulate bone formation than injection of exogenous growth factors.

FIGS. 16A-16C are fluorescent microscopy images showing results of a series of experiments in which groups of animals were exposed to PDGF or periosteal stimulation in presence of orthodontic transverse forces. Fluorescent microscopy demonstrates that PDGF in presence of orthodontic forces was able to slightly increase the rate of bone formation, but this effect was very apparent in presence of periosteal stimulation according to methods of the present invention (FIGS. 16A and 16B). To evaluate if injection of exogenous growth factor can have synergic effects on endogenous release of growth factor, in another series of experiments, animals were weekly exposed to both periosteal stimulation according to methods of the present invention and PDGF injection in presence of orthodontic forces. Fluorescent microscopy of these animals demonstrates significant increase in rate of bone formation in response to periosteal stimulation according to methods of the present invention, and demonstrates synergic effect between external and endogenous growth factors (FIG. 16C).

### DETAILED DESCRIPTION OF THE INVENTION

One aspect of the present invention relates to a device. The device includes an elongate handle having a proximal end and a distal end; a head portion extending from the proximal end of the elongate handle, the head portion having a first interior-facing surface and an opposing second exterior-facing surface; and a plurality of microneedles extending from, and perpendicular to, the first interior-facing surface of the head portion. Each of the plurality of microneedles has a base connected to the first interior-facing surface of the head portion and a free tip end.

The device according to the present invention is sometimes referred to herein as device, appliance, periosteal stimulator, periosteal stimulator appliance, bone growth stimulator, or cortical stimulator. It will be understood that such terms each refer to a device according to the present invention.

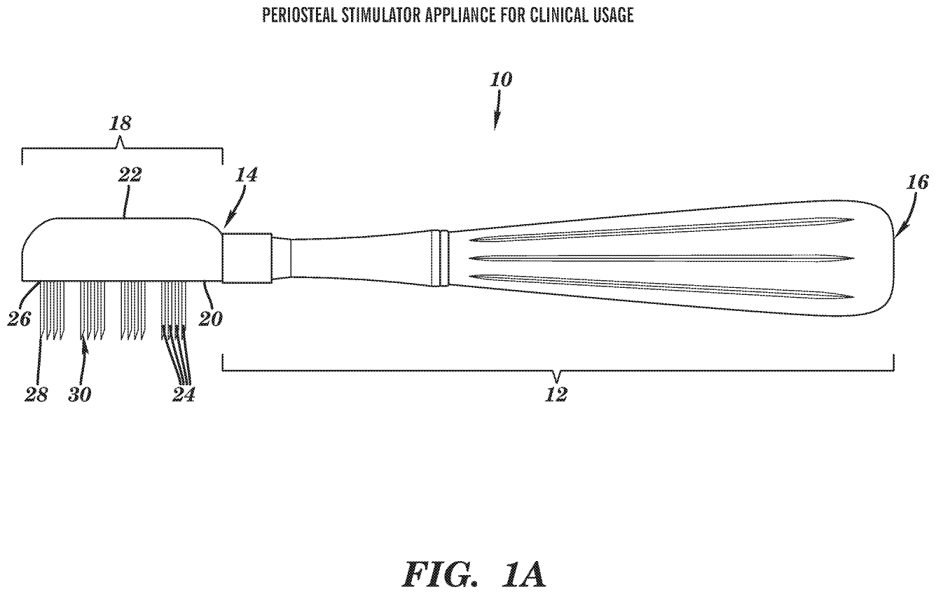

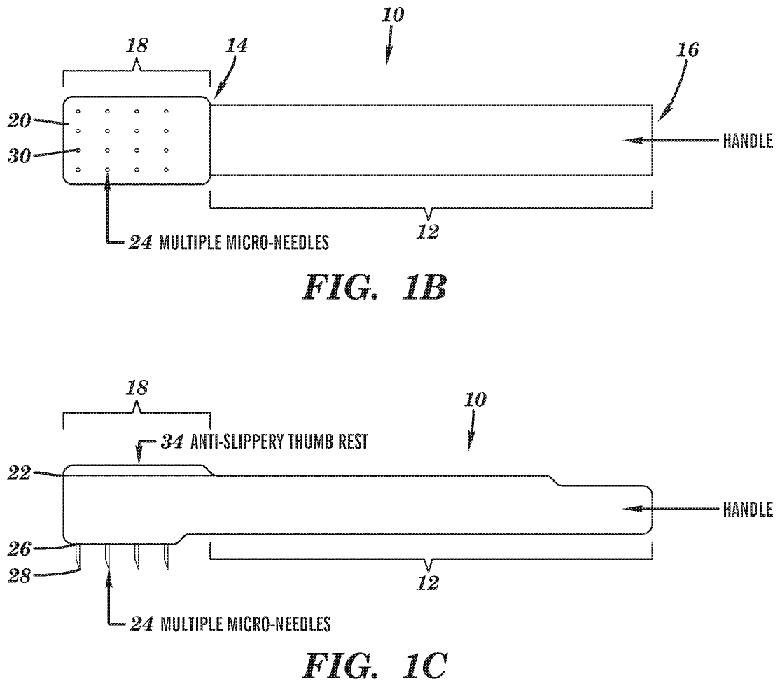

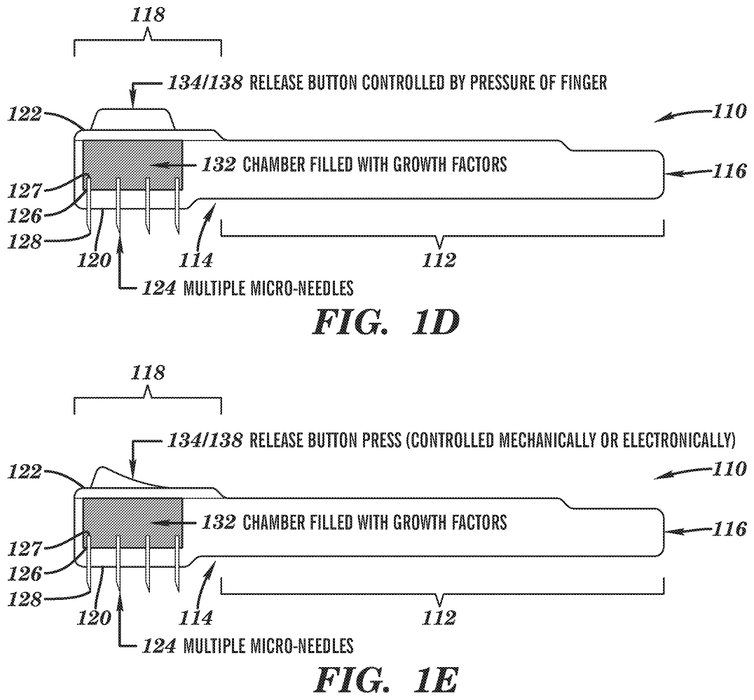

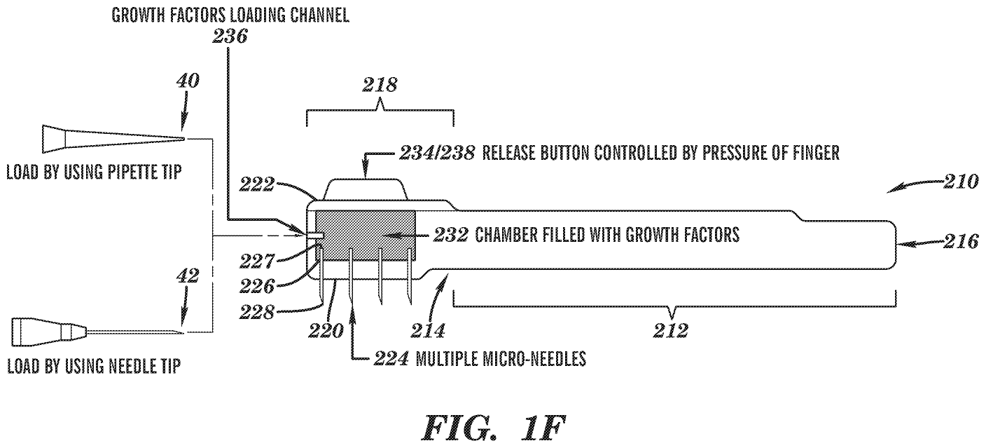

FIGS. 1A-1F are schematic illustrations of embodiments of a device according to the present invention that may be used to, for example, stimulate release of endogenous growth factors around the periosteum under the gum as described herein. This cortical bone stimulation device, in contrast to MOP devices (i.e., micro-osteoperforation devices), does not include a portion (e.g., a cutting tip) suitable to penetrate deeply (or at all) into bone. The device of the present invention may be used by a clinician manually under minimum local or topical anesthesia, in areas of interest or treatment, as described in detail below. Like numbers refer to like elements throughout.

With reference to FIGS. 1A-1F, the cortical bone stimulation device 10,110, 210 includes an elongate handle 12, 112, 212 having proximal end 14, 114, 214 and a distal end 16, 116, 216. Handle 12, 112, 212 may be a unitary piece or include multiple separable (or detachable) portions that are joined together (e.g., by threaded engagement (screwing), snapping, or the like) to form elongate handle 12, 112, 212. Elongate handle 12, 112, 212 may be of any length suitable for easy hand grasp by a user (e.g., clinician). Although shown with a flared design in FIG. 1A, it will be understood that elongate handle 12, 112, 212 may be of any suitable shape allowing easy grasp.

A head portion 18, 118, 218 extends from the proximal end 14, 114, 214 of handle 12, 112, 212. Head portion 18, 118, 218 and handle 12, 112, 212 may be unitary or separable (detachable) portions that are joined together (e.g., by threaded engagement (screwing), snapping, or the like) to form cortical bone stimulation device 10, 110, 210. Head portion 18, 118, 218 has a first interior-facing surface 20, 120, 220 and an opposing second exterior-facing surface 22, 122, 222. In one embodiment, the device is a dental device and head portion 18, 118, 218 is adapted for intra-oral placement in a subject's mouth. Head portion 18, 118, 218 may be of any suitable shape including, but not limited to round, ovoid, or rectangular.

In one embodiment, the exterior-facing surface 22, 122, 222 of the head portion 18, 118, 218 includes a rest 34, 134, 234 (see FIGS. 1C-1F) adapted to receive a thumb or forefinger. The rest 34, 134, 234 may be formed of an elastomeric material and/or any other anti-slip material. Rest 34, 134, 234 may also include projections or the like to prevent slip during use.

A plurality of microneedles 24, 124, 224 extend from, and are perpendicular to, the first interior-facing surface 20, 120, 220 of head portion 18, 118, 218. Each of the plurality of microneedles 24, 124, 224 has a base 26, 126, 226 connected to the first interior-facing surface 20, 120, 220 and a free tip end 28, 128, 228. Cortical bone stimulation device 10, 110, 210 may include, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 27, 38, 39, 40, 41, 42, 43, 44, 45, 46, 47, 48, 49, 50, and so on up to 100 or more microneedles 24, 124, 224. Each of the microneedles 24, 124, 224 has an exterior length from the base 26, 126, 226 to free tip end 28, 128, 228. As will be understood from reference to, for example, FIGS. 1D, 1E, and 1F, each of the microneedles 24, 124, 224 may have a further portion or length terminating at an interior end 27, 127, 227 (see, e.g., FIGS. 1D, 1E, 1F) within the device to, for example, stabilize or secure each of the microneedles 24, 124, 224 and/or provide for communication or fluidic coupling to fluid reservoir or compartment 132, 232. Each of the microneedles 24, 124, 224 may have a length from the base 26, 126, 226 to free tip end 28, 128, 228 of 0.1 mm to 10 mm. For instance, the length from the base 26, 126, 226 to free tip end 28, 128, 228 may be 0.1-1, 0.1-2, 0.1-3, 0.1-4, 0.1-5, 0.1-6, 0.1-7, 0.1-8, 0.1-9, 0.1-10, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, 9-10, 1, 2, 3, 4, 5, 6, 7, 8, 9, or 10 mm. The plurality of microneedles 24, 124, 224 may all have the same length from the base 26, 126, 226 to the free tip end 28, 128, 228. Alternatively, the plurality of microneedles 24, 124, 224 may have varying lengths relative to one another from the base 26, 126, 226 to the free tip end 28, 128, 228. For example, one or more of the plurality of microneedles may have a different length (from the base to the free tip end) than one or more of the other of the plurality of microneedles. Such variation permits the device to, e.g., contour to the cortical plate of a subject. In addition, plurality of microneedles 24, 124, 224 may be regularly or irregularly spaced on first interior-facing surface 20, 120, 220 of head portion 18, 118, 218. Plurality of microneedles 24, 124, 224 may be aligned in a plurality of regularly or irregularly spaced rows on first interior-facing surface 20, 120, 220 of head portion 18, 118, 218. Each of said microneedles 24, 124, 224 may include a bore 30, 130, 230 (see FIGS. 1A and 1B). As will be readily appreciated, bore 30, 130, 230 is a hollow passage running the entire length of each of said plurality of microneedles 24, 124, 224.

Plurality of microneedles 24, 124, 224 may be retractable within head portion 18, 118, 218 such that in a retracted position, each of the plurality of microneedles 24, 124, 224 is fully or partially contained in head portion 18, 118, 218. The plurality of microneedles 24, 124, 224 may be actuated from an extended to a retracted position (and a retracted position to an extended position) using mechanical or electrical actuation mechanism. The exterior length from base 26, 126, 226 to free tip end 28, 128, 228 of plurality of microneedles 24, 124, 224 may also be adjusted in this way, by actuating to a partially extended position using mechanical or electrical actuation mechanism.

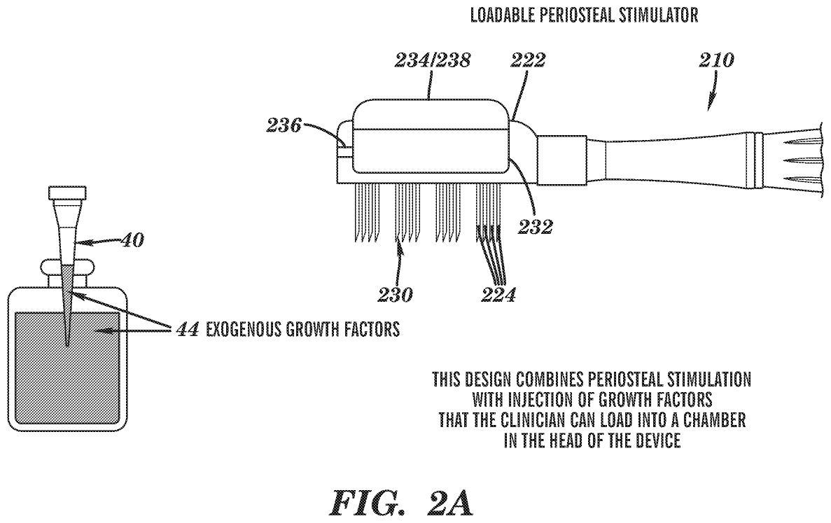

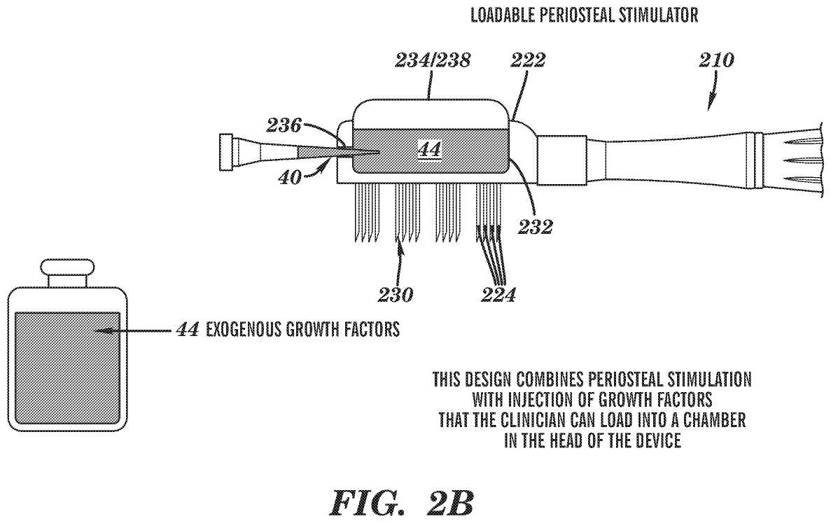

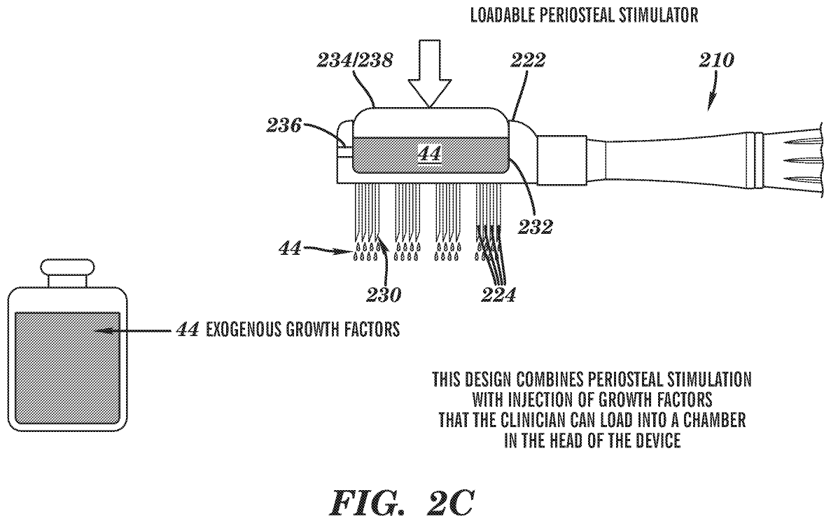

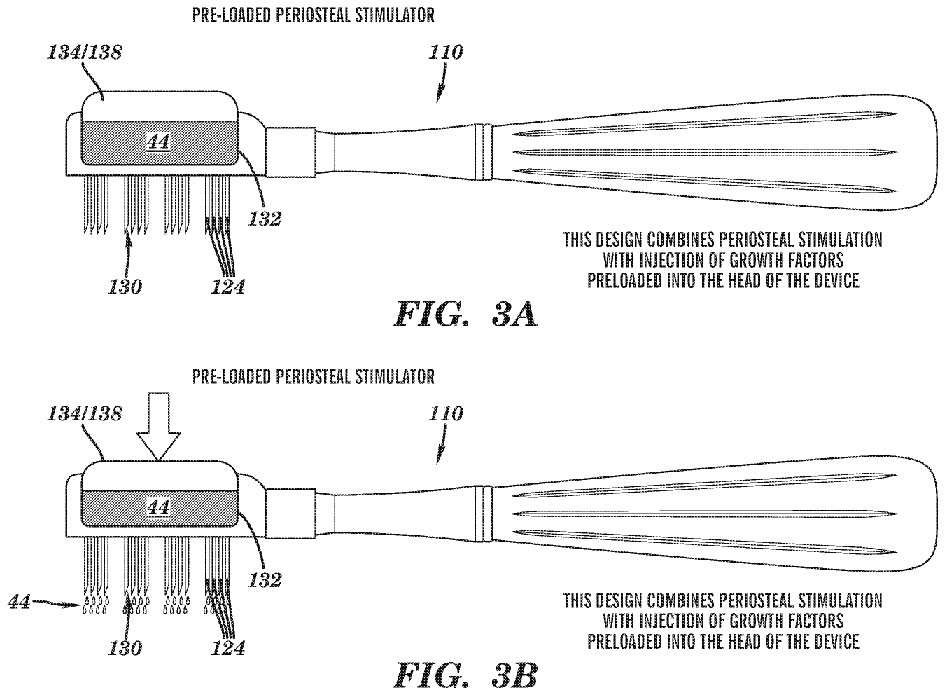

With reference to FIGS. 1D-1F, 2A-2C, 3A, and 3B the cortical bone stimulation device 110, 210 according to the present invention may also be adapted to include a fluid reservoir or compartment 132, 232 suitable to retain a composition for delivery to a subject's tissue through the plurality of microneedles 124, 224. For instance, the device 110, 210 according to the present invention may be used as described herein below to simultaneously deliver a composition 44 including, for example, exogenous growth factors to the area of application (i.e., are of tissue penetration) to take advantage of synergetic cortical bone stimulating effects of exogenous factors and endogenous factors. In this embodiment, fluid reservoir or compartment 132, 232 is in communication with bore 130, 230 of each of the plurality of microneedles 124, 224. The communication between fluid reservoir or compartment 132, 232 and bore 130, 230 of each of the plurality of microneedles 124, 224 is such that a composition 44 contained within fluid reservoir or compartment 132, 232 may be expelled through bore 130, 230 of each of the plurality of microneedles 124, 224, forming a fluidic pathway between fluid reservoir or compartment 132, 232 and bore 130, 230. In this embodiment, the cortical bone stimulation device 110, 210 may also include a release mechanism 138, 238, whereby actuation of the release mechanism releases contents of the fluid reservoir or compartment 132, 232 into the bore 130, 230 of and out the free tip end 128, 228 of each of the microneedles 124, 224. The release mechanism can be any of those known in the art (e.g., those known for use with glucose testing devices (see U.S. Patent 9,463,463, which is hereby incorporated by reference in its entirety)) and may be manually, mechanically, or electronically actuated. In one embodiment, the exterior-facing surface 122, 222 of the head portion 118, 218 includes the release mechanism 138, 238. In one embodiment, rest 134, 234 serves as the release mechanism 138, 238, whereby manual pressure is applied to rest/release mechanism 134/138, 234/238 to force the contents of the fluid reservoir or compartment 132, 232 into the bore 130, 230 of and out the free tip end 128, 228 of each of the microneedles 124, 224, as shown in FIGS. 2C, 3A, and 3B (arrows representing application of force or pressure to rest 134, 234). Rest/release mechanism 134/138, 234/238 may be in the form of a push button or other type of button, operated by manual pressure, mechanical trigger or switch, or electronical trigger or switch, or the like to inject the contents of the fluid reservoir or compartment 132, 232 into the subject's tissue.

With reference to FIGS. 1F and 2A-2C, the device 210 may also include port or channel 236 in communication with fluid reservoir or compartment 232. Port or channel 236 is adapted to receive, e.g., a tip of pipette 40 or needle 42 to facilitate filling or loading of device 210 with a composition. Also contemplated are embodiments like the one shown in FIGS. 1D, 1E, 3A, and 3B that do not include port or channel 236. Such embodiments may be pre-filled or pre-loaded with a composition including, e.g., exogenous growth factors, during manufacture. Also contemplated are pre-filled inserts (e.g., disposable inserts) that head portion 118, 218 is adapted to receive. Such inserts would contain a composition to be delivered and fit within fluid reservoir or compartment 132, 232.

Cortical bone stimulation device 10, 110, 210 may also include a removable cap affixed to interior-facing surface 20, 120, 220 of said head portion and covering the free-tip end 28, 128, 228 of each of the plurality of microneedles 24, 124, 224.

The device 10, 110, 210 according to the present invention or portions thereof (e.g., head portion 18, 118, 218 or elongate handle 12, 112, 212) may be fully disposable. For example, in one embodiment, the head portion 18, 118, 218 may be separable from the elongate handle 12, 112, 212 with the head portion 18, 118, 218 intended for single-use. The device 10, 110, 210 according to the present invention or portions thereof (e.g., head portion 18, 118, 218 or elongate handle 12, 112, 212) may be also made of a material suitable for sterilization and reuse.

Another aspect of the present invention relates to a method for stimulation of cortical bone formation. The method involves providing a device according to the present invention, as described in detail above. The device includes an elongate handle having a proximal end and a distal end; a head portion extending from the proximal end of the elongate handle, the head portion having a first interior-facing surface and an opposing second exterior-facing surface; and a plurality of microneedles extending from, and perpendicular to, the first interior-facing surface of the head portion. Each of the plurality of microneedles has a base connected to the first interior-facing surface of the head portion and a free tip end. The method also involves positioning the free tip end of said plurality of microneedles in contact with tissue of a subject, where cortical bone underlies the subject's tissue. The method further involves applying pressure to the device to cause the microneedles to penetrate the subject's tissue without penetrating the cortical bone underlying the subject's tissue, thereby stimulating formation of cortical bone underlying the subject's tissue.

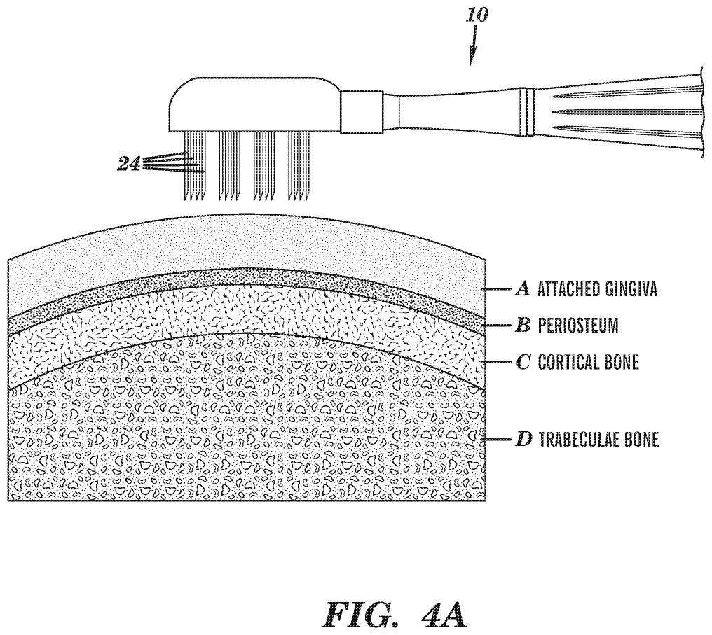

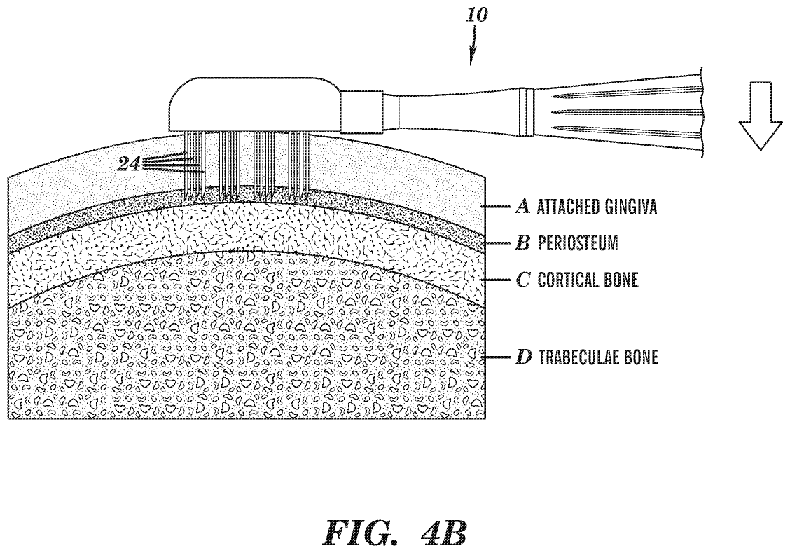

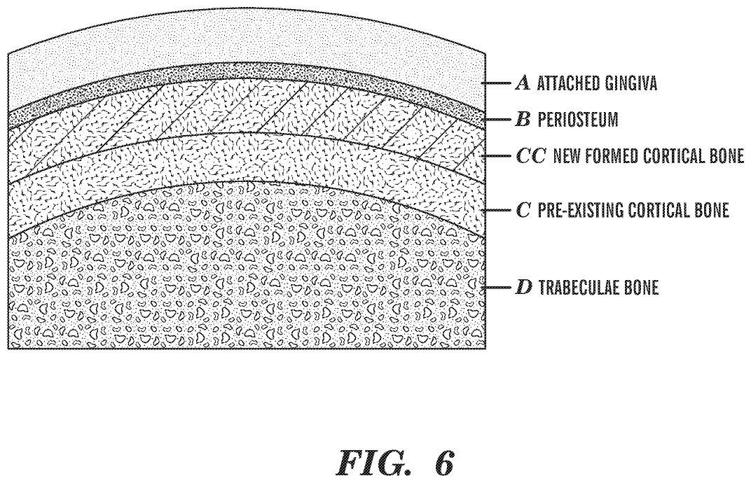

In one embodiment, the subject's tissue is penetrated by the microneedles includes gingival tissue, periosteal tissue, or both. With reference to FIGS. 4A-4D, in one embodiment the cortical bone is craniofacial bone and the cortical bone stimulation device 10 according to the present invention is positioned relative to the area of interest having attached gingiva A and periosteum B with underlying cortical bone C (FIG. 4A). Also shown is the trabeculae bone D that underlies cortical bone C. Pressure is applied to the device 10 in the direction of the large arrow shown in FIG. 4B suitable to cause microneedles 24 to penetrate the subject's soft tissue without penetrating underlying cortical bone (FIGS. 4B and 4C) and the device 10 is then withdrawn (FIG. 4D). This penetration of the soft tissue (i.e., attached gingiva A and periosteum B) without penetration of the underlying cortical bone C stimulates local release of endogenous growth factors under the periosteum (FIGS. 4C and 4D), which in turn stimulates new cortical bone growth CC (FIG. 6).

As shown in FIG. 4D, the method according to the present invention stimulates endogenous production of growth factors. Endogenous growth factors include (but are not limited to) osteogenic, osteoclastogenic, or angiogenic factors. See, e.g., U.S. Pat. No. 8,328,876 to Behnam et al., which is hereby incorporated by reference in its entirety. Such growth factors may include, but are not limited to, one or more insulin-like growth factors (IGF) (e.g., IGF-1 or IGF-2), platelet-derived growth factor (PDGF), fibroblast growth factors (FGF), vascular endothelial growth factor (VEGF), bone morphogenetic proteins (BMP) (e.g., BMP-2, BMP-3, BMP-4, BMP-5, BMP-6, BMP-7, BMP-8, BMP-9, BMP-10, BMP-11, BMP-12, and BMP-13), bone induction protein (BIP), transforming growth factors (TGF) (e.g., TGF-β), interleukins (e.g., IL-1β, IL-6, IL-8, IL-11, and IL-17), monocyte chemoattractant protein 1 (MCP-1 or CCL2), RANK ligand (RANKL), macrophage colony-stimulating factor (M-CSF), tumor necrosis factor alpha (TNFα), cathepsin K, or any other factor that stimulates or induces osteoclastogenesis or osteogenesis.

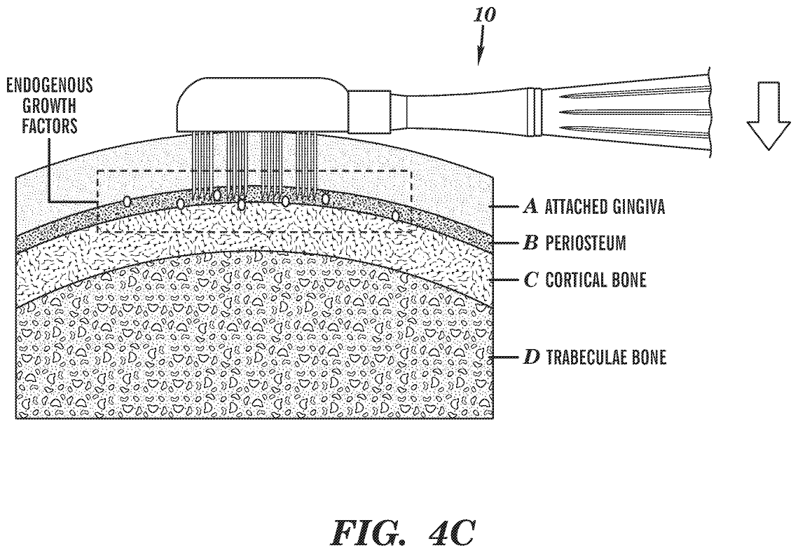

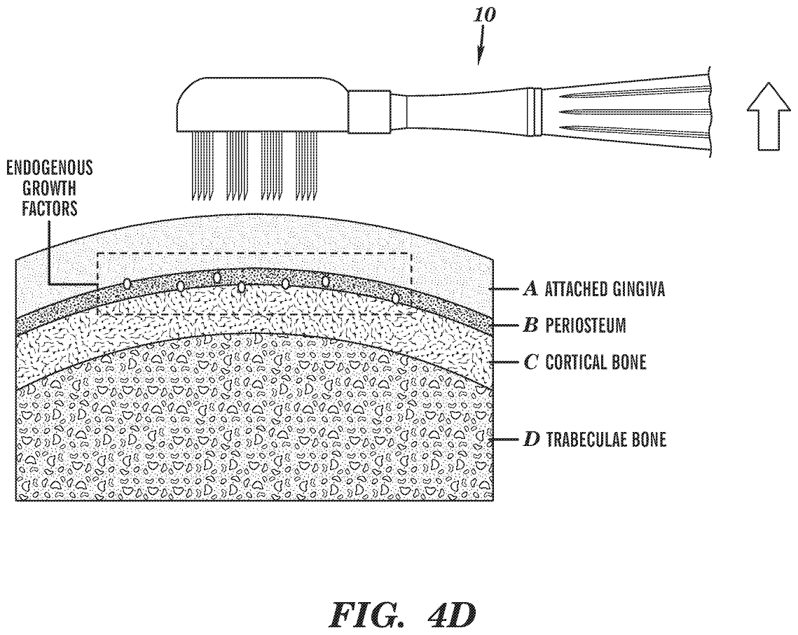

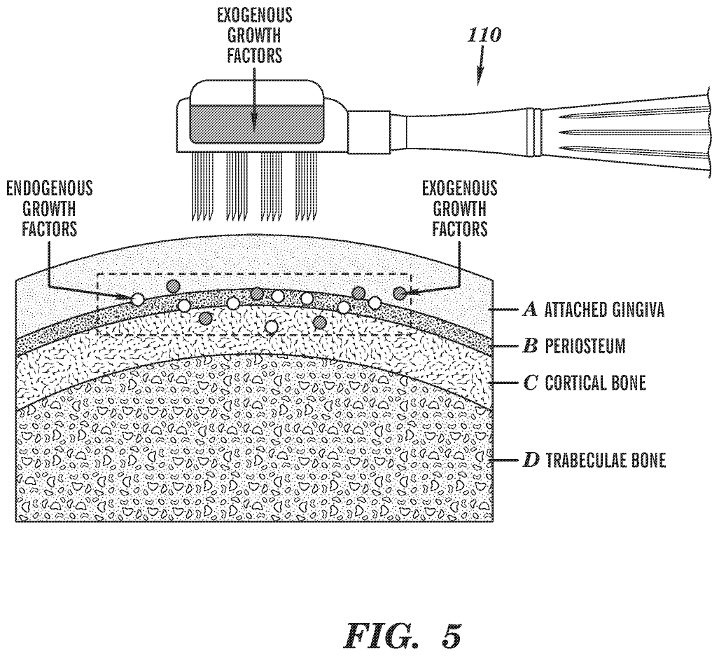

Although shown with respect to device 10 in FIGS. 4A-4D, it will be understood that the method according to the present invention may be carried out using any device described herein (including, e.g., device 110, 210 (see, e.g., FIG. 5)). The method may involve use of the cortical bone stimulation device 110, 210 according to the present invention to deliver or inject a composition into the subject's tissue that has underlying cortical bone. For instance, in one embodiment, the method is carried out with a cortical bone stimulation device 110, 210 as shown in FIGS. 1D-1F, 2A-2C, 3A, 3B, and 5. The fluid reservoir 132, 232 may include a composition. The composition may include one or more exogenous growth factors. Suitable exogenous growth factors include (but are not limited to) osteogenic, osteoclastogenic, or angiogenic factors. See, e.g., U.S. Pat. No. 8,328,876 to Behnam et al., which is hereby incorporated by reference in its entirety. Such exogenous growth factors include, but are not limited to, one or more insulin-like growth factors (IGF) (e.g., IGF-1 or IGF-2), platelet-derived growth factor (PDGF), one or more Fibroblast Growth Factors (FGF), vascular endothelial growth factor (VEGF), bone morphogenetic proteins (BMP) (e.g., BMP-2, BMP-4, BMP-5, BMP-6, BMP-10, BMP-11, BMP-12, and BMP-13), bone induction protein (BIP), one or more transforming growth factors (TGF) (e.g., TGF-β), interleukins (e.g., IL-1β, IL-6, IL-8, IL-11, and IL-17), monocyte chemoattractant protein 1 (MCP-1 or CCL2), RANK ligand (RANKL), macrophage colony-stimulating factor (M-CSF), tumor necrosis factor alpha (TNFα), cathepsin K, or any other factor that stimulates or induces osteoclastogenesis or osteogenesis.

In one embodiment, the method involves use of a device having a head portion including an exterior port defining a passage in communication with the fluid reservoir. This method may also involve delivering a composition through the exterior port and into the fluid reservoir. For example, as shown in FIGS. 1F and 2A-2C, a composition including exogenous growth factors may be manually loaded into a fluid reservoir or compartment 232 within the device 210 (FIGS. 2A-2B) via exterior port (or channel) 236 suitable to receive a pipette tip 40 (shown), needle tip, or the like.

These embodiments facilitate injection of the composition (including, e.g., exogenous growth factors) to the area of interest by expelling the composition through bore 130, 230 of microneedles 124, 224 of the device 110, 210 (FIGS. 2C, 3A, and 3B). In one embodiment, the method involves actuating a release mechanism as described above, whereby actuation of the release mechanism releases contents of the fluid reservoir or compartment 132, 232 into the bore 130, 230 and out the free tip end 128, 228 of each of the microneedles 124, 224. In one embodiment, the exterior-facing surface 122, 222 of the head portion 118, 218 includes the release mechanism. In one embodiment, the exterior-facing surface 122, 222 of the head portion 118, 218 includes a rest 134, 234 adapted to receive a thumb or forefinger. The rest 134, 234 may be formed of an elastomeric material. In one embodiment, the rest 134, 234 serves as the release mechanism, whereby manual pressure is applied to rest/release mechanism 134/138, 234/238 to force the contents 44 of the fluid reservoir or compartment 132, 232 into the bore 130, 230 of and out the free tip end 128, 228 of each of the microneedles 124, 224, as shown in FIGS. 2C, 3A, and 3B (arrows representing application of force or pressure to rest/release mechanism 134/138, 234/238).

With reference to FIG. 6, the method according to the present invention results in growth of newly formed cortical bone CC at the surface of the pre-existing cortical bone C. Although illustrated with respect to the dentofacial region, it will be appreciated that the method described herein may be used to stimulate cortical bone formation in other regions where cortical bone underlies soft tissue. For example, the methods and devices described herein may be used in any craniofacial region to cause cortical bone drift in a desired direction of soft tissue stimulation to, for example, restore bone shape in bone deficient or deformed areas. The methods described herein may also be used to drift cortical bone for cosmetic purpose to, e.g., improve facial aesthetics.

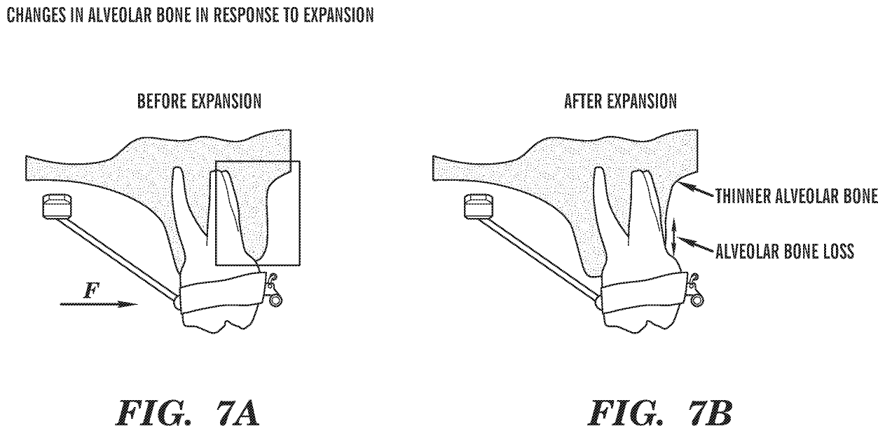

In one embodiment, the method according to the present invention is effective to induce osteogenesis or increase the rate of osteogenesis in cortical bone, as compared to when the method is not carried out. For example, FIGS. 7A and 7B are a schematic representations of changes in alveolar bone before expansion (FIG. 7A) and after expansion (FIG. 7B), illustrating of the effect of transverse orthodontic forces (F) on cortical bone in the absence of periosteal stimulation.

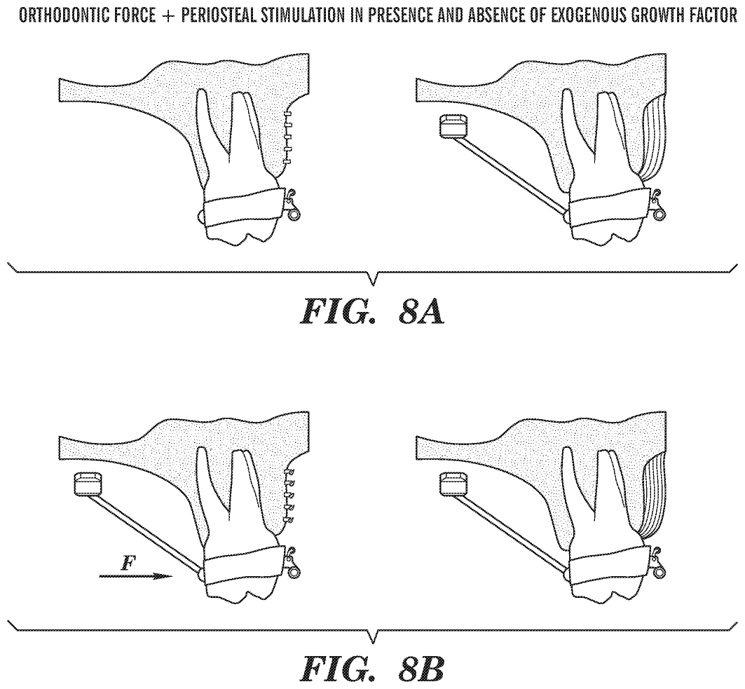

FIGS. 8A and 8B are schematic illustrations of the effect of orthodontic force (F) and periosteal stimulation according to the method of the present invention (left panels of FIGS. 8A and 8B, small rectangles) in presence (FIG. 8B) and absence (FIG. 8A) of exogenous growth factor (left panel of FIG. 8B, teardrop shapes). FIG. 8A illustrates the effect of periosteal stimulation according to the present invention that stimulates endogenous release of growth factors on the surface of the cortical bone as the tooth moves in response to orthodontic forces. FIG. 8B illustrates the synergistic effect of periosteal stimulation according to the present invention that induces release of endogenous growth factors coupled with release of exogenous growth factors during application of orthodontic forces.

Accordingly, in one embodiment, the subject may be undergoing orthodontic treatment. For instance the subject may be undergoing treatment to effect tooth movement, jaw expansion, or correction of dentoalveolar discrepancies. In one embodiment, the subject has a defect in the cortical bone. The defect may be, for example, due to cleft palate, periodontal disease, or trauma.

Use of the method according to present invention may lessen the time the patient undergoes orthodontic treatment to move a tooth or expand a jaw from a first position to a second position, or to correct the dentoalveolar discrepancies, as compared to when the method is not carried out. The orthodontic treatment time may be lessened by at least about 5%, 10%, 15%, 20%, 25%, 30%, 35%, 40%, 45%, 50%, 55%, 60%, 65%, 70%, 75%, 80%, 85%, 90%, or more as compared to the length of time required to achieve the same result where the method is not carried out.

The method may also further include providing an orthodontic appliance on or near the tooth to be moved or jaw to be expanded to exert force on the tooth or jaw toward the desired position. The orthodontic appliance may be installed prior to and/or subsequent to carrying out the claimed method.

The subject may also be one that has received a graft, implant, prosthesis, or the like (e.g., bone graft, bone implant, bone prosthesis, or the like known in the art) that would benefit from enhanced bone integration within the body of the subject, as compared to when the method is not carried out. The graft may be from the patient (autograft), a donor (allograft), or artificial. Accordingly, the method may also further include providing and/or installing a graft, implant, or prosthesis within the area of bone stimulation according the method described herein to facilitate, e.g., integration of the graft, implant, or prosthesis. The graft, implant, or prothesis may be provided and/or installed prior to and/or subsequent to carrying out the claimed method. Use of the method according to present invention may lessen the time for integration of the graft, implant, prosthesis, or the like, as compared to when the method is not carried out. The integration time may be lessened by at least about 5%, 10%, 15%, 20%, 25%, 30%, 35%, 40%, 45%, 50%, 55%, 60%, 65%, 70%, 75%, 80%, 85%, 90%, or more as compared to the length of time required to achieve the same result where the method is not carried out.

The methods described herein may also include first administering anesthesia to the subject. The methods described herein may also include first administering a local or topical anesthesia to the subject.

The subject may be any mammal. In one embodiment, the subject is a human. The subject may be an adult, child, or adolescent.

The methods of the present invention may be repeated as necessary to cause sufficient cortical bone growth, as a clinician determines necessary. For instance, the methods may repeated daily, one, two, three, four, five, six, seven, or more times per week, or one, two, three, four, five, eight, ten, twelve, fifteen, twenty or more times per month for a duration of, e.g., 1 to 12 months.

Another aspect of the present invention is directed to a kit for providing the components for carrying out stimulation of cortical bone formation, the kit including one or more of, a local anesthetic, a topical anesthetic, a syringe for application of local anesthetic, and a device (or detachable head portion thereof) according to the present invention as described herein in a disposable package.

### EXAMPLES

### Example 1

### Periosteal Stimulation Causes Cortical Bone Formation

As noted above, there is an enormous need for a technique that can stimulate bone formation at the surface of cortical bone, non-invasively to be able to expand the limit of orthodontic dental corrections.

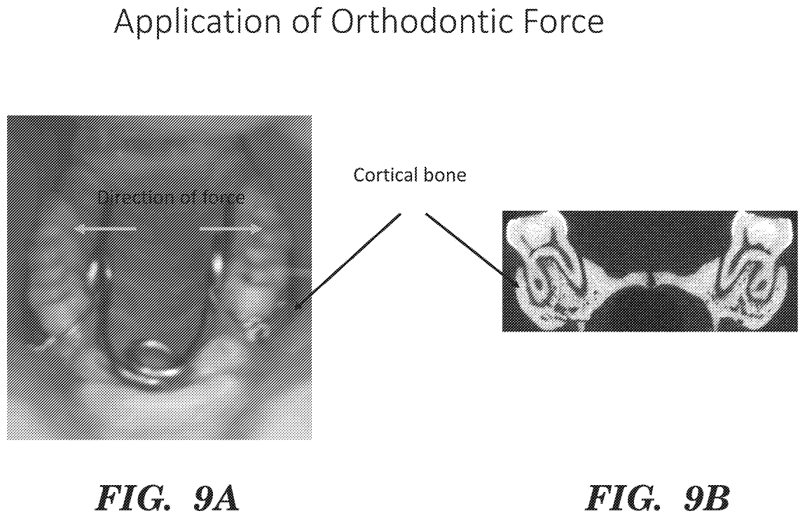

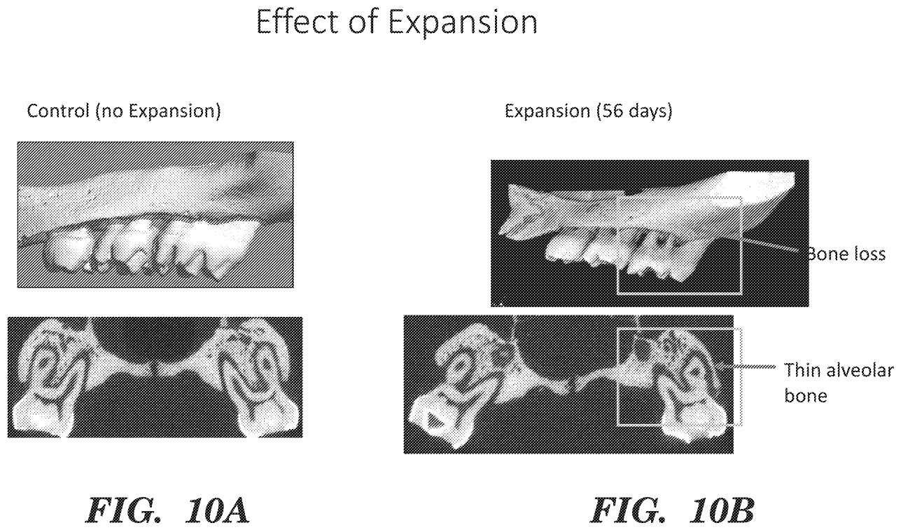

To address this challenge, in the first series of experiments, rats (5 per group) were exposed to orthodontic transverse forces, applied to the upper molars and pushing towards the cortical bone (FIGS. 9A and 9B). After 56 days the animal were sacrificed and micro-CT images were collected. FIGS. 10A and 10B are micro-CT images of a control (no expansion) (FIG. 10A) and expansion (FIG. 10B) subjects collected after 56 days. In response to orthodontic forces, teeth were moved towards the cortical bone (FIG. 10B). As shown, the bone did not follow the tooth and, therefore, bone loss around the roots of the tooth was apparent (FIG. 10B, top panel) and the cortical plate became thinner (FIG. 10B, bottom panel).

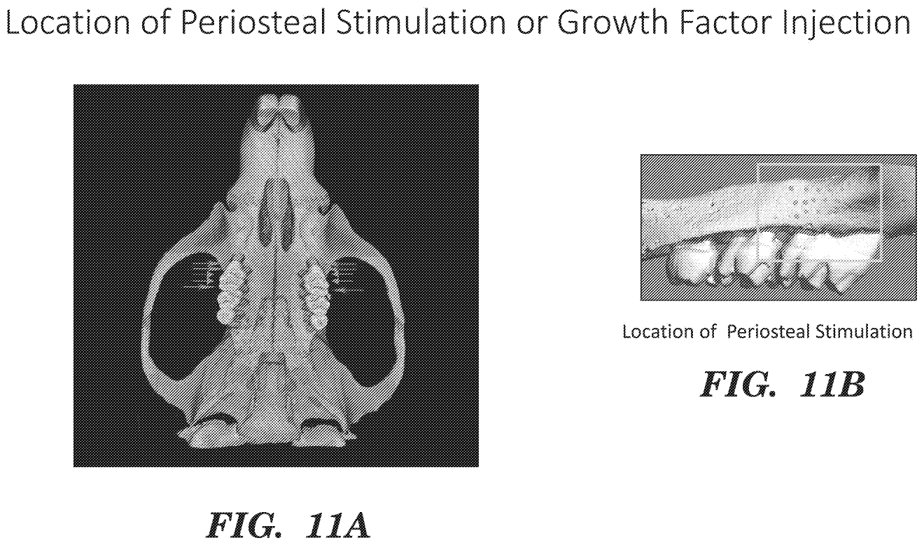

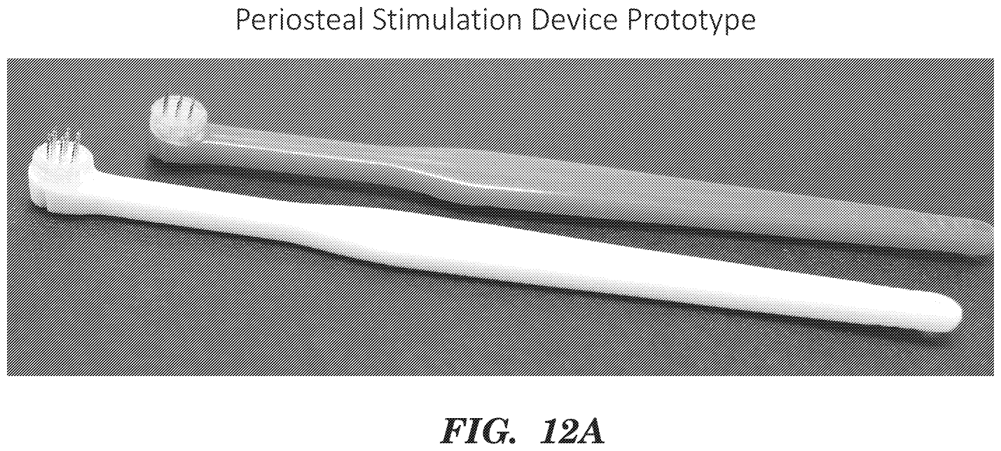

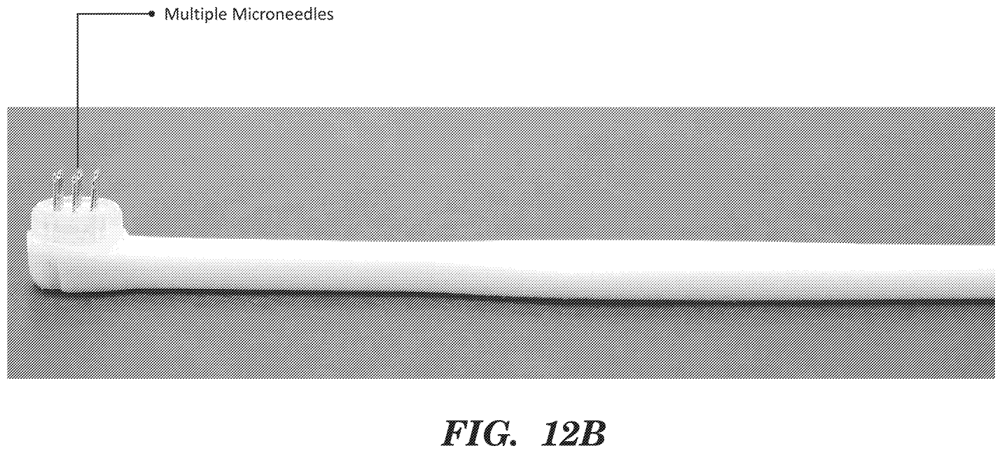

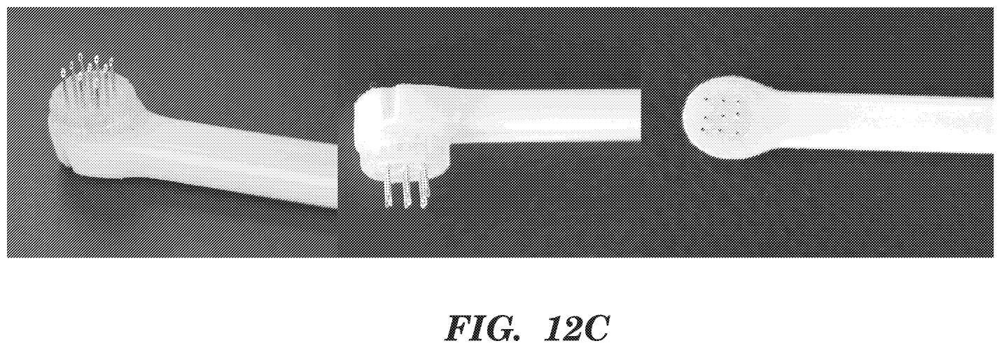

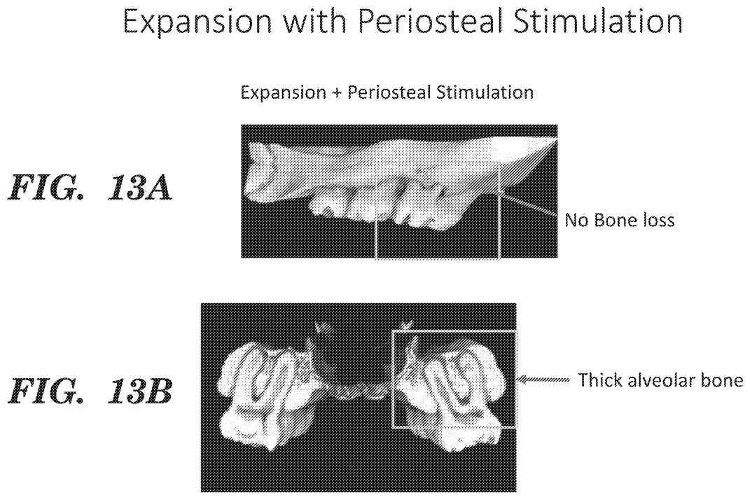

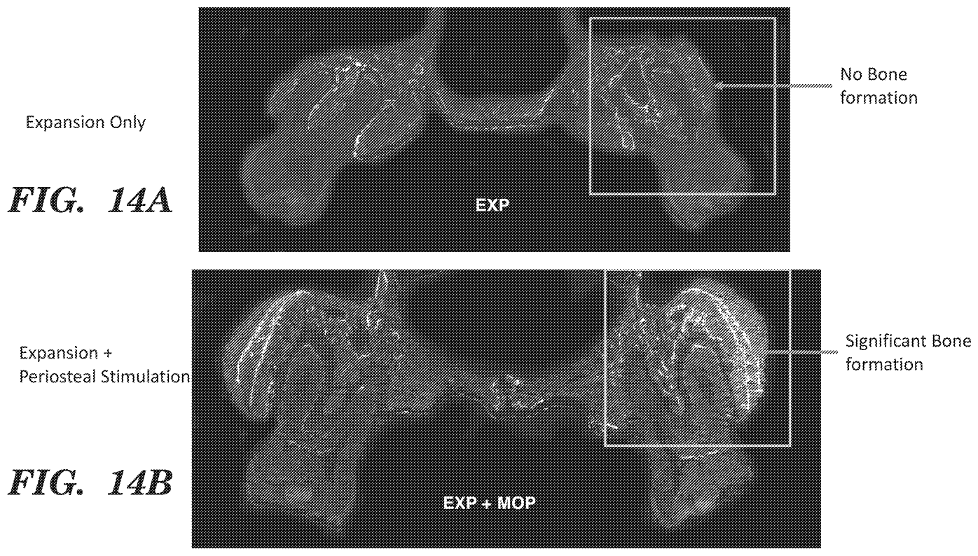

In a second experimental group, in addition to transverse orthodontic forces, animals were exposed to periosteal stimulation around the area of first and second molar (arrows in FIG. 11A and dots within the boxed area of FIG. 11B) once per week. Periosteal stimulation was carried out with an appliance similar to the one depicted in FIG. 12A-12C, with microneedles 3-5 mm in length. After 56 days of expansion treatment these animals did not show any bone loss, and in addition cortical bone did not get thinner (FIGS. 13A and 13B). Fluorescent microscopy demonstrates significant increase in rate of bone formation in the area of cortical bone, that was clinically very significant, and never possible before (FIGS. 14A and 14B).

### Example 2

### Periosteal Stimulation with Delivery of Exogenous Growth Factor Causes Synergistic Cortical Bone Formation

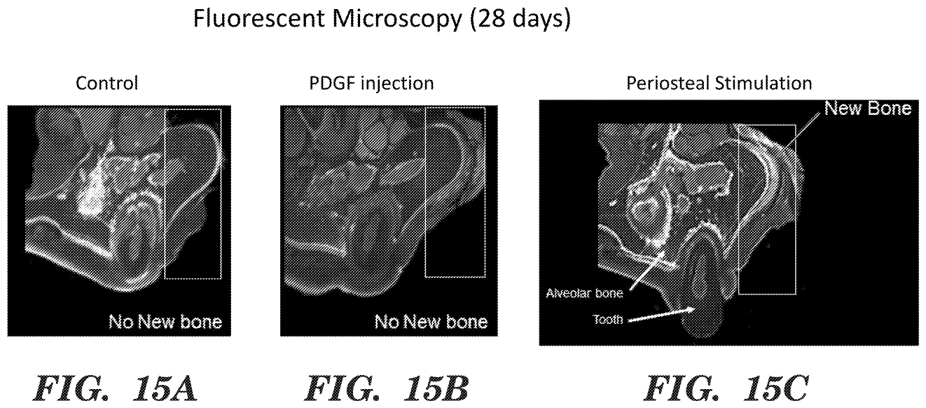

One possible reason for this stimulation may be the local release of growth factors in the area. Therefore, in another series of experiments, animals were divided into 3 groups: 1) control that did not receive any treatment; 2) animals that received injection of PDGF once per week (around first molar area); and 3) animals that received periosteal stimulation once per week in a similar area. No orthodontic force was applied to these animals/teeth. Compared to control (FIG. 15A), the group that received injection of PDGF did not show significant bone formation (FIG. 15B) while the group that received periosteal stimulation demonstrated significant bone formation (FIG. 15C). These results indicate that the endogenous release of growth factors in response to periosteal stimulation is more effective to stimulate bone formation than injection of exogenous growth factors.

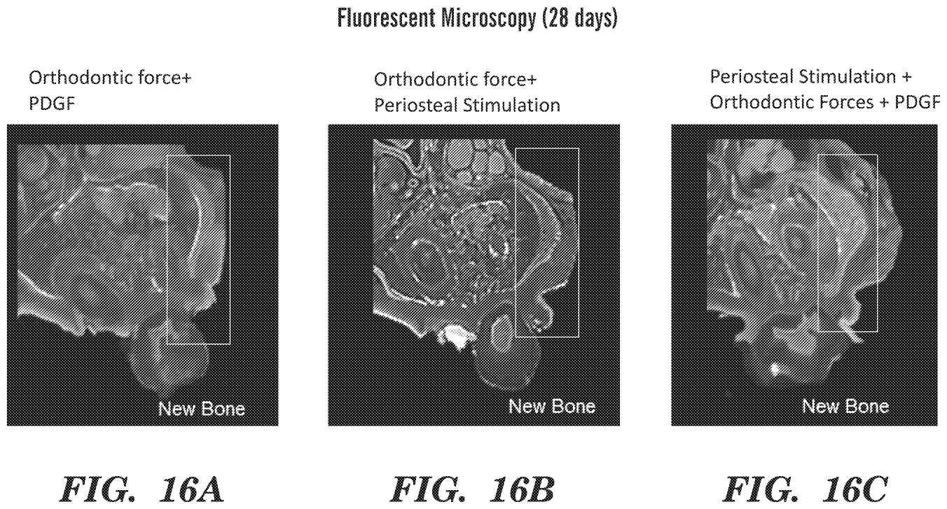

To see if this effect can accentuate the effect of bone remodeling that is stimulated by orthodontic forces, in another series of experiments, similar groups of animals were exposed to PDGF or periosteal stimulation in presence of orthodontic transverse forces. Fluorescent microscopy demonstrates that PDGF in presence of orthodontic forces was able to slightly increase the rate of bone formation, but this effect was very apparent in presence of periosteal stimulation (FIGS. 16A and 16B).

To evaluate if injection of exogenous growth factor can have synergic effects on endogenous release of growth factor, in another series of experiments, animals were weekly exposed to both periosteal stimulation and PDGF injection in presence of orthodontic forces. Fluorescent microscopy of these animals demonstrates significant increase in rate of bone formation in response to periosteal stimulation and demonstrates synergic effect between external and endogenous growth factors (FIG. 16C).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A device comprising:
an elongate handle having a proximal end and a distal end;
a head portion extending from the proximal end of said elongate handle, said head portion having a first interior-facing surface and an opposing second exterior-facing surface;
a plurality of microneedles extending from, and perpendicular to, the first interior-facing surface of said head portion, each of said plurality of microneedles having a base connected to the first interior-facing surface of said head portion and a free tip end.

2.-4. (canceled)

5. The device according to claim 1, wherein each of said plurality of microneedles has a length from base to tip end of 0.1 mm to 10 mm.

6. (canceled)

7. (canceled)

8. The device according to claim 1, wherein said plurality of microneedles are retractable within said head portion such that in a retracted position, each of said plurality of microneedles is fully contained in said head portion.

9. The device according to claim 1, wherein at least one of said plurality of microneedles comprises a bore and said head portion further comprises a fluid reservoir in communication with the bore and an exterior port in communication with said fluid reservoir.

10. (canceled)

11. (canceled)

12. The device according to claim 9, wherein said head portion further comprises a release mechanism, whereby actuation of the release mechanism releases contents of the fluid reservoir through into the bore and out the tip end of each of said one or more microneedles comprising the bore.

13. (canceled)

14. (canceled)

15. The device according to claim 1, wherein the exterior-facing surface of said head portion further comprises a rest adapted to receive a thumb or forefinger.

16.-18. (canceled)

19. The device according to claim 1, wherein the device further comprises:
a removable cap affixed to the interior-facing surface of said head portion and covering the free-tip end of said plurality of microneedles.

20. A method for stimulation of cortical bone formation, the method comprising:
providing a device comprising:
an elongate handle having a proximal end and a distal end;
a head portion extending from the proximal end of said elongate handle, the head portion having a first interior-facing surface and an opposing second exterior-facing surface;
a plurality of microneedles extending from, and perpendicular to, the first interior-facing surface of said head portion, each of said plurality of microneedles having a base connected to the first interior-facing surface of said head portion and a free tip end;
positioning the free tip end of said plurality of microneedles in contact with tissue of a subject, wherein cortical bone underlies the subject's tissue; and
applying pressure to the device to cause said microneedles to penetrate the subject's tissue without penetrating the cortical bone underlying the subject's tissue, thereby stimulating formation of cortical bone underlying the subject's tissue.

21. (canceled)

22. The method according to claim 20, wherein the subject's tissue comprises gingival tissue, periosteal tissue, or both.

23. The method according to claim 20, wherein the cortical bone is craniofacial cortical bone.

24. (canceled)

25. (canceled)

26. The method according to claim 20, wherein each of said plurality of microneedles has a length from base to tip end of 0.1 mm to 10 mm.

27. (canceled)

28. (canceled)

29. The method according to claim 20, wherein said plurality of microneedles are retractable within said head portion such that in a retracted position, each of said plurality of microneedles is fully contained in said head portion.

30. The method according to claim 20, wherein at least one of said plurality of microneedles comprises a bore and said head portion further comprises a fluid reservoir in communication with the bore and an exterior port defining a passage in communication with said fluid reservoir, and the method further comprises delivering a composition through the exterior port into the fluid reservoir.

31. (canceled)

32. The method according to claim 30, wherein the fluid reservoir comprises a composition.

33. (canceled)

34. The method according to claim 32, wherein the composition comprises one or more osteogenic, angiogenic, and/or osteoclastogenic factors selected from the group of insulin-like growth factors (IGF), platelet-derived growth factor (PDGF), fibroblast growth factors (FGF), vascular endothelial growth factor (VEGF), bone morphogenetic proteins (BMP), bone induction protein (BIP), transforming growth factors (TGF), interleukins, monocyte chemoattractant protein 1 (MCP-1 or CCL2), RANK ligand (RANKL), macrophage colony-stimulating factor (M-CSF), tumor necrosis factor alpha (TNFα), or cathepsin K.

35. (canceled)

36. The method according to claim 30, wherein said head portion further comprises a release mechanism, and wherein the method further comprises actuating the release mechanism and thereby releasing contents of the fluid reservoir into the bore and out the tip end of each of said one or more microneedles comprising the bore.

37.-40. (canceled)

41. The method according to claim 20, wherein the device further comprises a removable cap affixed to the interior-facing surface of said head portion and covering the free-tip end of said plurality of microneedles and wherein said method further comprises removing said removable cap prior to said positioning.

42. The method according to claim 20, wherein said applying is effective to increase endogenous production of one or more osteogenic, angiogenic, and/or osteoclastogenic factors in the subject's tissue compared to when the method is not carried out selected from the group of insulin-like growth factors (IGF), platelet-derived growth factor (PDGF), fibroblast growth factors (FGF), vascular endothelial growth factor (VEGF), bone morphogenetic proteins (BMP), bone induction protein (BIP), transforming growth factors (TGF), interleukins, monocyte chemoattractant protein 1 (MCP-1 or CCL2), RANK ligand (RANKL), macrophage colony-stimulating factor (M-CSF), tumor necrosis factor alpha (TNFα), or cathepsin K.

43. (canceled)

44. The method according to claim 20, wherein said applying is effective to induce or increase the rate of osteogenesis in cortical bone underlying the subject's tissue, as compared to when said applying is not carried out.

45.-47. (canceled)

48. The method according to claim 20, wherein the subject is undergoing orthodontic treatment to effect tooth movement, jaw expansion, or correction of dentoalveolar discrepancies.

49. (canceled)

50. The method according to claim 20, wherein the subject has a defect in the cortical bone due to cleft palate, periodontal disease, or trauma.

51.-54. (canceled)
